(19) United States
(12) Patent Application Publication
(10) Pub. No.: US 2011/0279268 A1
(43) Pub. Date: Nov. 17, 2011

(54) MEDICAL APPARATUS

(75) Inventors: SUMIHITO KONISHI, TOKYO (JP); KOTA SUGAYA, TOKYO (JP)

(73) Assignee: OLYMPUS MEDICAL SYSTEMS CORP., TOKYO (JP)

(21) Appl. No.: 13/080,059

(22) Filed: Apr. 5, 2011

Related U.S. Application Data
(63) Continuation of application No. PCT/JP2010/068044, filed on Oct. 14, 2010.
(60) Provisional application No. 61/255,539, filed on Oct. 28, 2009.

(51) Int. Cl. A61N 7/00; G08B 21/00

(52) U.S. Cl. 340/540; 601/2

(57) ABSTRACT

An ultrasound output apparatus includes: a priority level judgment section for judging a priority level for preferentially notifying a user of a plurality of abnormalities that occur in the plurality of handpieces; a display control section for causing a display section to display abnormality information whose priority level judged by the priority level judgment section is high; and a priority level changing section, which upon detecting an occurrence of an abnormality from a handpiece, which is different from the handpiece in which the abnormality information being displayed on the display section is originated, causes abnormality information from the hand piece to be displayed on the display section in place of the abnormality information being displayed on the display section regardless of the priority level of the abnormality.

### CROSS REFERENCE TO RELATED APPLICATION

This application is a continuation application of PCT/JP2010/068044 filed on Oct. 14, 2010 and claims benefit of U.S. Provisional Patent Application No. 61/255,539 filed in the U.S.A. on Oct. 28, 2009, the entire contents of which are incorporated herein by this reference.

### BACKGROUND OF THE INVENTION

1. Field of the Invention

The present invention relates to a medical apparatus, and particularly to a medical apparatus including a display section for displaying abnormality states of a medical device.

2. Description of the Related Art

In recent years, ultrasound output apparatuses for giving treatment by using ultrasound energy, or high-frequency output apparatuses (hereafter, also referred to as an electrosurgical knife) for giving treatment by a high-frequency current to a target living tissue etc. have become widely used.

An ultrasound output apparatus has a display section for displaying setting information of a medical device to be connected thereto. A user confirms the setting information which is displayed on the display section to grasp the operation state of the medical device.

When an abnormality occurs in the medical device, an error code corresponding to the abnormality which has occurred in the medical device is displayed on the display section to notify that to the user. Further, it is configured that when a plurality of abnormalities simultaneously occur in a medical device, an error code is displayed according to a predetermined display rule to notify that to the user.

For example, Japanese Patent Application Laid-Open Publication No. 2005-681 discloses a control system in which when a plurality of abnormalities occur in the same device, an error code of a higher importance level, which is predetermined, is displayed.

### SUMMARY OF THE INVENTION

The medical apparatus of an embodiment of the present invention is a medical apparatus connectable with a plurality of medical devices, the medical apparatus comprising: a priority level judgment section for judging a priority level for preferentially notifying a user of a plurality of abnormalities that occur in the plurality of medical devices; a display control section for causing a display section to display abnormality information whose priority level judged by the priority level judgment section is high; and a priority level changing section, which upon detecting an occurrence of an abnormality from a medical device other than the medical device in which the abnormality information being displayed on the display section is originated, causes abnormality information from the medical device to be displayed on the display section in place of the abnormality information being displayed on the display section regardless of the priority level of the abnormality.

### BRIEF DESCRIPTION OF THE DRAWINGS

FIG. 1 is a diagram to illustrate the configuration of a surgical system relating to an embodiment of the present invention;

FIG. 2 is a front view of an ultrasound output apparatus seen from the front;

FIG. 3A is a block diagram showing the internal configuration of a high-frequency output apparatus;

FIG. 3B is a block diagram showing the internal configuration of an ultrasound output apparatus;

FIG. 4 is a diagram showing the electric configuration of a handpiece connector and an output connector;

FIG. 5 is a diagram to illustrate the relationships among priority levels, abnormality details, and occurrence categories;

FIG. 6 is a diagram to illustrate an example of a display screen when the handpieces 2a and 2b are connected to the ultrasound output apparatus 4;

FIG. 7A is a diagram to illustrate an example of the error display screen which is displayed on the display section 60 when an ESG-400 communication abnormality occurs in the handpiece 2a;

FIG. 7B is a diagram to illustrate an example of the countermeasure information display screen which is displayed on the display section 60;

FIG. 8 is a diagram to illustrate an example of an error display screen which is displayed on the display section 60 when an ultrasound amplitude abnormality occurs in the handpiece 2b;

FIG. 9 is a diagram to illustrate an example of the error display screen which is displayed on the display section 60 when an apparatus abnormality occurs in the ultrasound output apparatus 4;

FIG. 10 is a diagram to illustrate an example of the error display screen which is displayed on the display section 60 when a probe damage abnormality occurs in the handpiece 2a;

FIG. 11 is a diagram to illustrate an example of an all-instruments display screen 80 in which setting information is black out;

FIG. 12 is a flowchart to illustrate an example of the flow of the processing to cause an error display screen to be displayed on the display section 60;

FIG. 13 is a diagram to illustrate an example of backup data;

FIG. 14 is a diagram to illustrate an example of the switching rule to perform output operation of the ultrasound output apparatus or the high-frequency output apparatus in the case of standalone operation in the present embodiment; and

FIG. 15 is a diagram to illustrate an example of the switching rule relating to the output operation in the case of combined operation of the ultrasound output apparatus and the high-frequency output apparatus in the present embodiment.

### DETAILED DESCRIPTION OF THE PREFERRED EMBODIMENT

Hereafter, embodiment of the present invention will be described in detail with reference to the drawings.

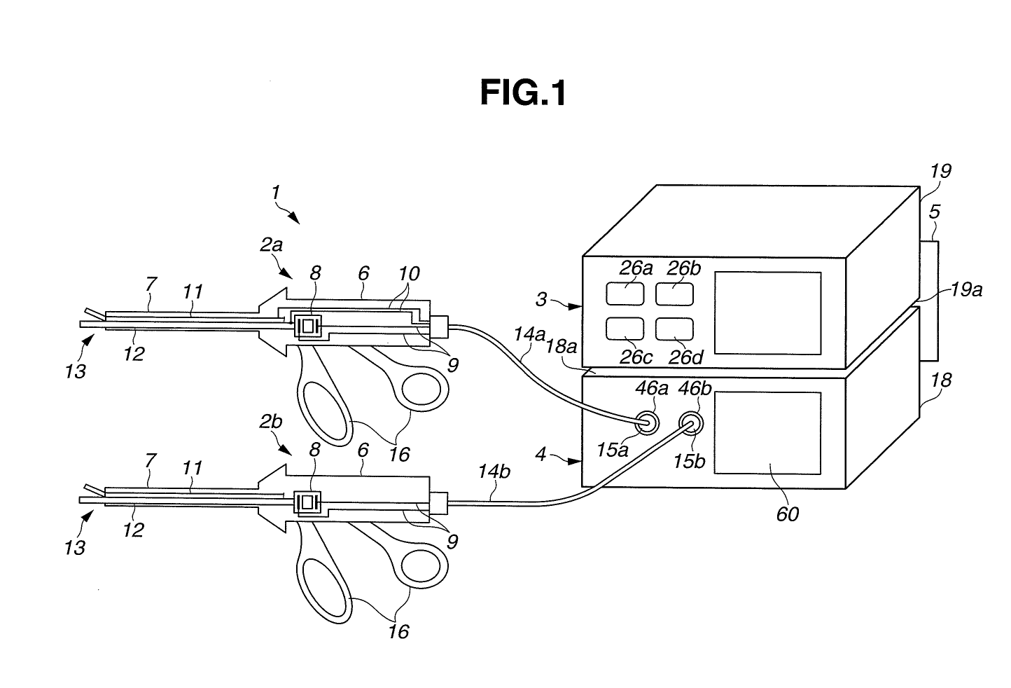

As shown in FIG. 1, a surgical system 1 of an embodiment of the present invention is configured to include handpieces 2a and 2b, a high-frequency output apparatus 3, and an ultrasound output apparatus 4.

The high-frequency output apparatus 3 and the ultrasound output apparatus 4 are connected with each other via a below described docking connector. Moreover, the high-frequency output apparatus 3 and the ultrasound output apparatus 4 are connected with each other by, for example, a communication cable 5 in the back side.

The high-frequency output apparatus 3 outputs a high-frequency signal to the handpiece 2a via the docking connector and the ultrasound output apparatus 4.

The ultrasound output apparatus 4 as a medical apparatus outputs an ultrasound driving signal that causes a below described ultrasound transducer 8, which is contained in each of the handpieces 2a and 2b, to undergo ultrasound vibration.

The handpieces 2a and 2b are medical devices for respectively giving treatment to a target living tissue. In particular, the handpiece 2a is a medical device that simultaneously outputs ultrasound and high-frequency wave to give treatment to a target living tissue, and the handpiece 2b is a medical device that outputs only ultrasound to give treatment to a target living tissue.

The handpiece 2a has two output modes including a sealing and cutting mode (hereafter, also referred to as a SEAL & CUT mode) that simultaneously outputs an ultrasound and a high-frequency wave, and a sealing mode (hereafter, also referred to as a SEAL mode) that outputs only a high-frequency wave. These two modes are switched by a hand switch which is not shown and provided in a below described grasping portion 6 of the handpiece 2a.

Moreover, the handpiece 2b has two output modes including a maximum output mode (hereafter, also referred to as a MAX mode) that maximizes and outputs ultrasound, and a variable output mode (hereafter, also referred to as a VAR mode) that variably outputs ultrasound. These two modes are switched by a hand switch which is not shown and provided in a below described grasping portion 6 of the handpiece 2b.

The handpiece 2a includes a grasping portion 6 which is grasped and operated by an operator, and a sheath portion 7 that is extended forward from the grasping portion 6. The grasping portion 6 and the sheath portion 7 contain an ultrasound transducer 8, an ultrasound cable 9, a high-frequency cable 10, a wire 11, and a probe 12.

Note that in the distal end side of the probe 12, a treatment portion 13 is formed of a distal end portion of the probe 12 and a mobile piece that operates so as to be openable and closable with respect to the distal end portion.

Moreover, the distal end of the cable 14a is connected to the rear end of the grasping portion 6. A handpiece connector (hereafter, referred to as an HP connector) 15a is provided in the rear end of the cable 14a, and the HP connector 15a is removably connected to an output connector 46a of the ultrasound output apparatus 4.

The ultrasound cable 9 and the high-frequency cable 10 are inserted into the cable 14a, the rear ends of the ultrasound cable 9 and the high-frequency cable 10 are connected to the output connector 46a of the ultrasound output apparatus 4 via the HP connector 15a.

The ultrasound output apparatus 4 allows the supply of an ultrasound driving signal to the ultrasound transducer 8 within the grasping portion 6 via the ultrasound cable 9 in the cable 14a. Accordingly, the supply of an ultrasound driving signal causes the ultrasound transducer 8 to undergo ultrasound vibration. The ultrasound transducer 8 is transferred to the distal end portion of the probe 12 via the probe 12 in the sheath portion 7. Thus, the handpiece 2a can cause a target living tissue to generate friction heat by the ultrasound vibration energy, thereby giving treatment such as sealing and cutting.

Moreover, two high-frequency cables 10 that transmit a high-frequency signal are inserted into the cable 14a, and one of distal ends of the high-frequency cables 10 is connected to the rear end of the probe 12, and the other is connected to the rear end side of the wire 11 that is in conduction with the movable piece. Note that the movable piece, the probe 12, and the wire 11 are made up of a conductor such as a metal that transmits a high-frequency signal. It may be configured that the high-frequency cable 10 is connected to the movable piece with a lead wire inserted into the sheath portion 7 without being connected to the rear end side of the wire 11. Thus, the handpiece 2a can give treatment of high-frequency cautery by applying a high-frequency current into a living tissue grasped by the treatment portion 13.

Moreover, the grasping portion 6 is provided with a finger engaging portion 16 for performing opening and closing operation. The operator can perform opening and closing operation with its finger in engagement with the finger engaging portion 16 to pull the wire 11 inserted into the sheath portion 7 so that the movable piece in the treatment portion 13 can be opened or closed, thereby grasping a target living tissue.

In the meantime, in contrast to the handpiece 2a, the handpiece 2b is configured not to include a high-frequency cable 10 because it outputs only ultrasound. Other components are similar to those of the handpiece 2a and therefore description thereof will be omitted.

The distal end of a cable 14b is connected to the rear end of the grasping portion 6 of the handpiece 2b. An HP connector 15b at the rear end of a cable 14b is removably connected to an output connector 46b of the ultrasound output apparatus 4.

The ultrasound cable 9 is inserted into the cable 14b, and the rear end of the ultrasound cable 9 is connected to the output connector 46b of the ultrasound output apparatus 4 via the HP connector 15b.

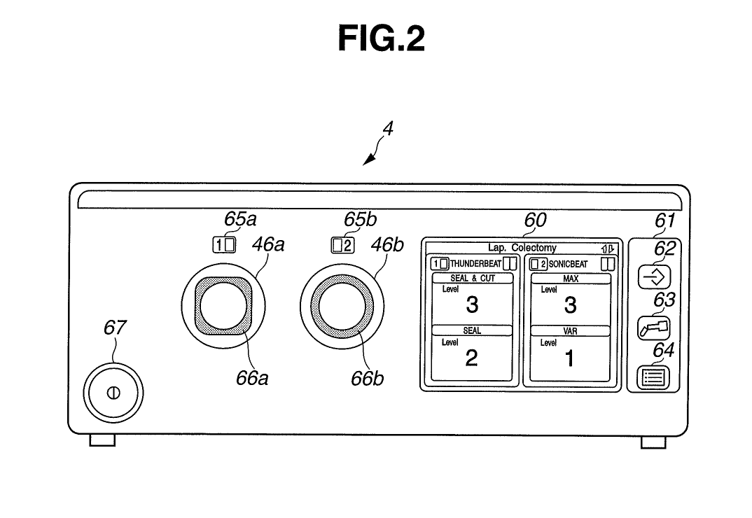

Next, the configuration of the front of the ultrasound output apparatus 4 will be described by using FIG. 2. FIG. 2 is a front view of the ultrasound output apparatus 4 seen from the front.

As shown in FIG. 2, the ultrasound output apparatus 4 is provided with output connectors 46a and 46b, a display section 60, a sheet switch 61, symbols 65a and 65b, and a power supply button 67. Moreover, the sheet switch 61 is provided with a selection button 62, a probe check button 63, and a menu button 64. The output connectors 46a and 46b are provided with flange receivers 66a and 66b, respectively.

The display section 60 displays a below described error display screen, etc. The display section 60 is configured such that a touch panel is disposed on a liquid crystal display, and a user can, for example, change settings of output level etc. by touching the display screen with a finger or a dedicated pen.

The probe check button 63 is a button for performing a probe check. As described above, the handpiece 2a needs to perform a probe check with a conductive material interposed between the movable piece and the probe 12 to simultaneously output high-frequency wave and ultrasound. In the present embodiment, the probe check button 63 performs only ultrasound output to perform a probe check. This obviates the need of performing check with a conductive material being interposed between the movable piece and the probe 12.

The menu button 64 is a button for causing a menu screen for performing various settings such as sound volume setting etc. to be displayed. A user can press the menu button 64 thereby causing the menu screen to be displayed on the display section 60.

The symbols 65a and 65b are respectively provided corresponding to the output connectors 46a and 46b at the front of the ultrasound output apparatus 4. A symbol similar to the symbol 65a is provided by printing, for example, in the grasping portion 6 of the handpiece 2a. Moreover, a symbol similar to the symbol 65b is provided by printing, for example, in the grasping portion 6 of the handpiece 2b. This allows the operator to confirm the symbol provided in the grasping portion 6 of the handpiece 2a and recognize to which of the output connectors 46a and 46b the HP connector 15a is to be connected. As a result of this recognition, the operator is prevented from erroneously connecting the HP connector 15a to the output connector 46b.

Moreover, the ultrasound output apparatus 4 is provided with a plurality of (two in this case) output connectors 46a and 46b. When the two output connectors 46a and 46b have the same shape, only providing the above described symbols 65a and 65b may lead to a risk of erroneous mounting of the HP connector.

Accordingly, a substantially rectangular flange not shown is provided in the HP connector 15a, and the substantially rectangular flange is adapted to fit into the flange receiver 66a of the output connector 46a. Moreover, the HP connector 15b is provided with a substantially circular flange not shown, and the substantially circular flange is adapted to fit into the flange receiver 66b of the output connector 46b. This allows the prevention of erroneous mounting between the HP connector and the output connector.

Note that it may be configured that flange receivers 66a and 66b have different colors to make the shapes of the flange receivers 66a and 66b more conspicuous.

The power supply button 67 is a button for turning on or off the power supply of the ultrasound output apparatus 4.

Moreover, as described above, a touch panel is disposed on the liquid crystal display in the display section 60. In general, the apparatus including a touch panel has a calibration function for performing positional correction of the touch panel in preparation for the case in which the position of the touch panel is deviated. When performing the calibration, the user operates the touch panel to make a transition to a calibration screen and executes the calibration. In this case, when the position of the touch panel is significantly deviated, it may be unable to make a transition to the calibration screen.

Therefore, the ultrasound output apparatus 4 has a function to make a direct transition to the calibration screen. In the present embodiment, it is configured such that turning on the power supply button 67 while simultaneously pressing a selection button 62 and the probe check button 63 of the sheet button 61 allows the transition to the calibration screen. Thus, even when the position of the touch panel is significantly deviated, the positional correction of the touch panel can be performed.

Moreover, when an abnormality occurs, the ultrasound output apparatus 4 can cause various log data for performing abnormality analysis to be displayed on the display section 60. When an abnormality occurs in the ultrasound output apparatus 4, however, the touch panel may become inoperable and it may become unable to confirm various log data for performing abnormality analysis.

Therefore, the ultrasound output apparatus 4 has a function to make a direct transition to the screen of various log data. The present embodiment is configured such that turning on the power supply button 67 while simultaneously pressing the probe check button 63 and the menu button 64 of the sheet button 61 allows direct transition to the screen of various log data. Thus, even when the touch panel is inoperable, it is possible to confirm various log data for performing abnormality analysis.

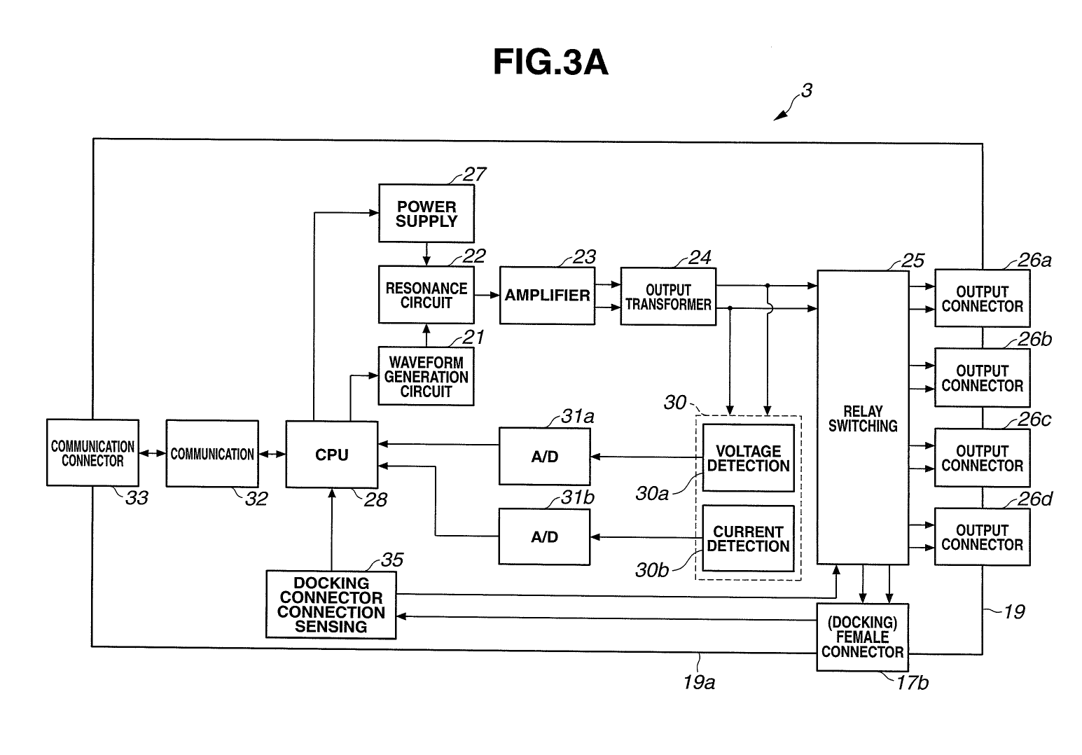

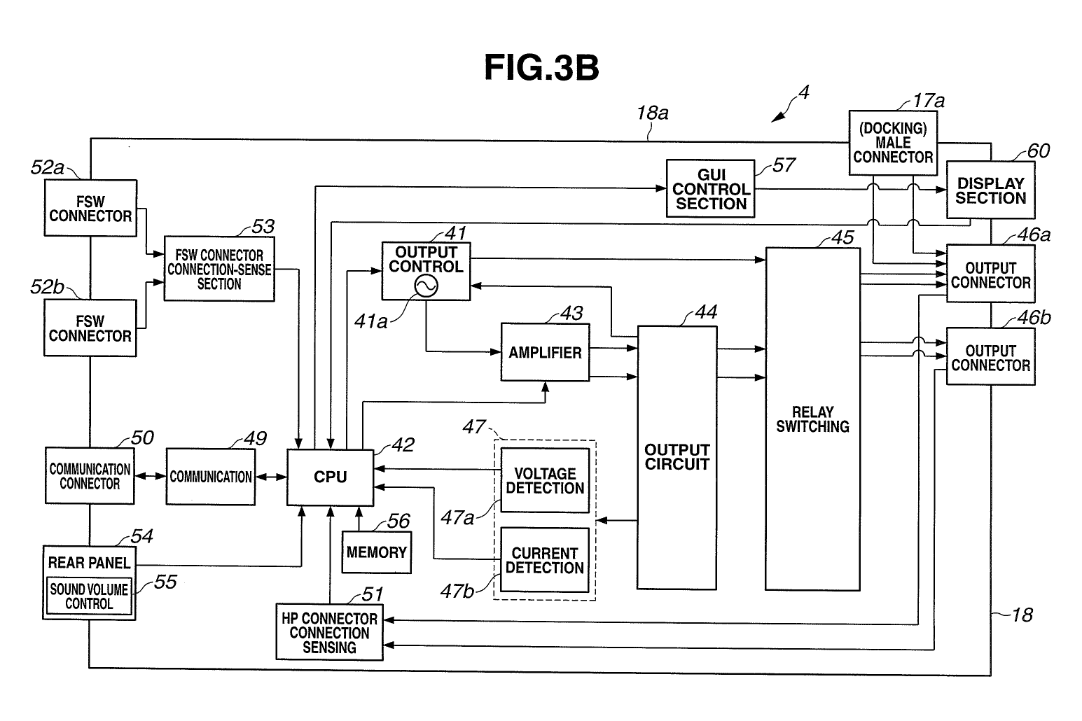

Next, the internal configuration of the high-frequency output apparatus 3 and the ultrasound output apparatus 4 will be described by using FIGS. 3A and 3B.

FIG. 3A is a block diagram showing the internal configuration of the high-frequency output apparatus 4. FIG. 3B is a block diagram showing the internal configuration of the ultrasound output apparatus 4.

First, the configuration of a docking connector will be described.

A docking male connector (abbreviated as a male connector) 17a that makes up a docking connector is provided on a top plate 18a of a housing 18 as a storage case of the ultrasound output apparatus 4.

Moreover, a docking female connector (abbreviated as a female connector) 17b that makes up the docking connector is provided in the bottom plate 19a of the housing 19 as a storage case of the high-frequency output apparatus 3. Thus, it is possible to dock the male connector 17a with the female connector 17b, which are respectively provided in opposed positions in the surfaces of both plates, to establish a connection of the connectors by placing the housing 19 of the high-frequency output apparatus 3 on the top plate 18a of the housing 18 of the ultrasound output apparatus 4.

The high-frequency signal that is outputted from the high-frequency output apparatus 3 is outputted from the female connector 17b provided in the bottom plate 19a of the housing 19 to the output connector 46a through the male connector 17a provided at an opposing position on the top plate 18a of the housing 18 of the ultrasound output apparatus 4. Thus, the high-frequency signal is transmitted to the handpiece 2a via the cable 14a connected to the HP connector 15a.

Next, the internal configuration of the high-frequency output apparatus 3 will be described. The high-frequency output apparatus 3 contains a waveform generation circuit 21 for generating a sinusoidal wave and a burst wave, and the signal of a sinusoidal wave or a burst wave outputted from this waveform generation circuit 21 is inputted to an amplifier 23 through a resonance circuit 22.

A signal amplified by the amplifier 23 is applied to the primary winding side of an output transformer 24, and a high-frequency signal for use in cautery is generated on the secondary winding side.

The secondary winding of the output transformer 24 is connected to, for example, four output connectors 26a, 26b, 26c, and 26d and the female connector 17b that makes up the docking connector via a relay switching circuit 25 which switches a high-frequency signal to be outputted.

Note that as described above, the female connector 17b is provided in the bottom plate 19a of the housing 19. Moreover, the resonance circuit 22 is supplied with a power supply voltage from a variable-voltage power supply circuit 27, and the waveform generation circuit 21 and the power supply circuit 27 are controlled by a CPU 28 as a control section.

The CPU 28 controls the waveform generation circuit 21 and the power supply circuit 27 in correspondence with the settings of the output mode by the setting section not shown and output set values, etc.

The output signal of the secondary winding of the above described output transformer 24 is inputted into a voltage detection circuit 30a and a current detection circuit 30b which make up a detection section 30.

The voltage detection circuit 30a and the current detection circuit 30b respectively measure, in other words, detect the voltage and current in a high-frequency signal outputted from the secondary winding of the output transformer 24. The detected voltage and current are converted into a digital voltage and current by A/D converters 31a and 31b, respectively and are inputted to the CPU 28.

The CPU 28 calculates, in other words, detects a high-frequency power from the inputted voltage and current as the product thereof. Then, the CPU 28 controls the voltage by the power supply circuit 27 such that the detected value of the high-frequency power becomes a set value preset by the above described setting section.

Moreover, the CPU 28 is connected with a communication connector 33 via a communication circuit 32 which performs communication. The communication connector 33 is connected with a communication connector 50 on the side of the ultrasound output apparatus 4 shown in FIG. 3B via the communication cable 5.

The above described female connector 17b which is connected with the relay switching circuit 25 is removably connected with the male connector 17a on the side of the ultrasound output apparatus 4 as described above.

Moreover, for example, two connector pins for connection sensing in the female connector 17b are connected with a docking connector connection-sense circuit 35, and the docking connector connection-sense circuit 35 constantly senses the connection between the male connector 17a and the female connector 17b by using the connector pins for connection sensing.

In this case, the two connector pins for connection sensing is specified to be connected, for example, with two short-circuited connector pins on the side of the other male connector 17a.

Thus, the docking connector connection-sense circuit 35 can perform connection sensing of whether or not the docking connectors 17 are connected, by sensing whether or not the two connector pins for connection sensing are in a conducting state.

Then, the connection sensing result by the docking connector connection-sense circuit 35 is transferred to the CPU 28. The CPU 28 inhibits the simultaneous output of ultrasound output and high-frequency output when the connection sensing result by the docking connector connection-sense circuit 35 is no-connection.

In other words, the CPU 28 permits the simultaneous output of ultrasound output and high-frequency output only when the connection of the docking connectors 17 is sensed.

Moreover, when having sensed a connection between the male connector 17a and the female connector 17b, the docking connector connection-sense circuit 35 controls the switching of the relay switching circuit 25 such that the output signal of the output transformer 24 is outputted to the female connector 17b side. Note that the CPU 28 may perform the control of switching instead of the docking connector connection-sense circuit 35.

In the meantime, the ultrasound output apparatus 4 shown in FIG. 3B includes an output control circuit 41 containing an oscillating circuit 41a. The oscillating circuit 41 adjusts the frequency and the current of the oscillation signal oscillated at the oscillating circuit 41a under the control of a CPU 42 as a control section.

The signal amplified by an amplifier 43 is inputted into an output circuit 44, and is amplified by a transformer, which is not shown, of the output circuit 44 to be outputted as an ultrasound driving signal from the secondary winding of the transformer.

The output connector 46a is also connected with the male connector 17a. The connector 46a is connected to the handpiece 2a that outputs ultrasound and high-frequency wave.

Note that the output connector 46b is not connected with the male connector 17a, but connected with an ultrasound specific handpiece 2b that outputs ultrasound independently from the high-frequency output apparatus 3.

The ultrasound driving signal outputted from the output circuit 44 is inputted into a voltage detection circuit 47a and a current detection circuit 47b which make up a detection section 47, and voltage and current are respectively measured, in other words, detected.

The detected voltage and current are inputted into the CPU 42 via A/D converters not shown inside the voltage detection circuit 47a and the current detection circuit 47b, respectively.

Moreover, the ultrasound output apparatus 4 is provided with a setting section not shown which sets the power of the ultrasound driving signal to be supplied to the ultrasound transducer 8 of the handpiece 2a, and the setting information thereof is inputted into the CPU 42.

The CPU 42 performs a constant current control through the output control circuit 41 based on the voltage and current detected through the detection section 47 such that power set by the setting section is outputted from the output circuit 44.

For this purpose, the control is performed such that the control information of the output value when being outputted from the output circuit 44 is temporarily retained in a memory in the output control circuit 41 so that the CPU 42 corrects the immediately preceding control information via the output control circuit 41 by the voltage and current that are detected thereafter.

Moreover, the CPU 42 is connected with a communication connector 50 via a communication circuit 49 that performs communication. The communication connector 50 is connected with a communication connector 33 on the side of the high-frequency output apparatus 3 shown in FIG. 3A via the communication cable 5. The CPU 42 and the CPU 28 can perform a bidirectional communication through the communication cable 5.

Note that the present embodiment includes in order to inhibit output when there is an abnormality in the communication path between the high-frequency output apparatus 3 and the ultrasound output apparatus 4: detection means such as a Network Management Vector or a Heart Beat that detects a disconnection of the communication instruction path including the communication line, the output circuit, output instruction means, and the communication line; and output inhibition means for inhibiting output when a disconnection is detected.

Moreover, connector connection-sense pins in the output connectors 46a and 46b are connected with an HP connector connection-sense circuit 51. The HP connector connection-sense circuit 51 senses connection or no-connection of each of the HP connectors 15a and 15b.

Note that the handpiece 2a is connected to the output connector 46a as described above, and the handpiece 2b is connected to the output connector 46b as described above. The HP connector connection-sense circuit 51 sends the information of sensing result to the CPU 42.

The CPU 42 controls the switching of the relay switching circuit 45 via the output control circuit 41 such that an output signal, that is, an ultrasound driving signal from the output circuit 44 is supplied to the output connecter to which the handpiece is connected, according to the information of the sensing result. Note that the CPU 42 may control the switching of the relay switching circuit 45.

Moreover, the ultrasound output apparatus 4 includes foot switch (hereafter, referred to as FSW) connectors 52a and 52b to which two foot switches not shown are connected. The FSW connectors 52a and 52b are connected to an FSW connector connection-sense section 53.

The FSW connector connection-sense section 53 senses whether or not the foot switches are connected respectively to the FSW connectors 52a and 52b, and outputs the sensing information to the CPU 42.

Moreover, a rear panel 54 is provided in the back of the ultrasound output apparatus 4. The rear panel 54 is provided with a sound volume control 55 so that a user can adjust the sound volume of the ultrasound output apparatus 4 by operating the sound volume control 55.

A memory 56 stores setting information which is used in the last time, that is, information such as an output level etc. Moreover, the memory 56 stores backup data to be described later. The CPU 42 reads out the information such as the output level etc. from the memory 56 and outputs it to a GUI control section 57.

The GUI control section 57 performs the control to cause a below described all-instruments display screen to be displayed on the display section 60 when the handpieces 2a and 2b are connected to the ultrasound output apparatus 4. Moreover, when different abnormalities have occurred in the handpieces 2a and 2b, the GUI control section 57 performs the control to cause an error display screen relating to the abnormality which occurs later to be displayed on the display section 60, as described later. Further, when different abnormalities occur in the ultrasound output apparatus 4 and the handpiece 2a or 2b, the GUI control section 57 performs the control to cause an error display screen relating to the abnormality which has occurred in the ultrasound output apparatus 4 to be displayed on the display section 60. Furthermore, when different abnormalities have occurred in the same apparatus, the GUI control section 57 performs control based on a priority level to cause an error display screen relating to the abnormality whose priority level is high to be displayed on the display section 60.

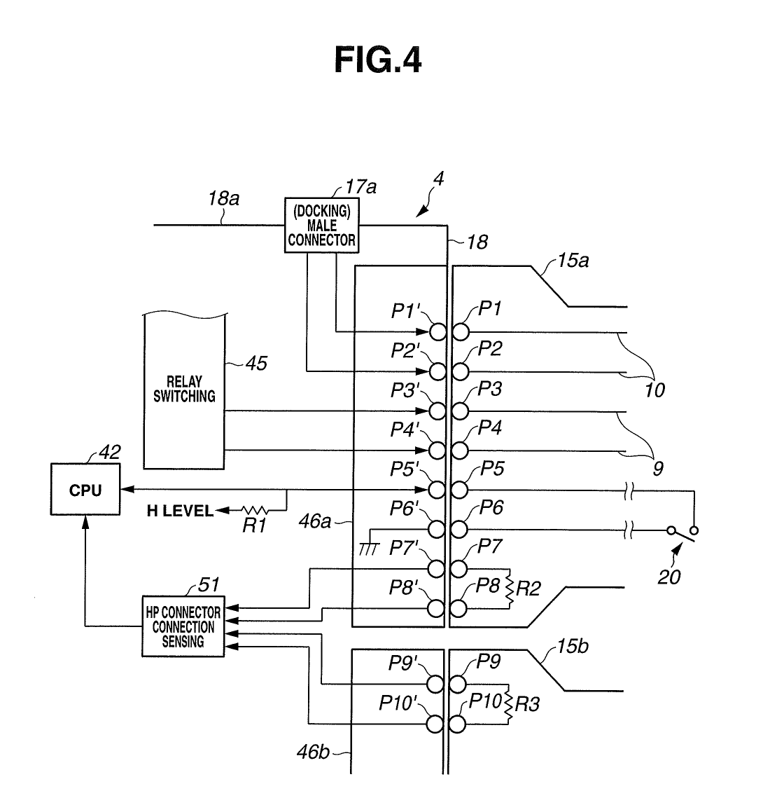

FIG. 4 is a diagram showing the electric configuration of the handpiece connector and the output connector. Connector pins P1 and P2 are connected to the male connector 17a respectively via the connector pins P1′ and P2′ of the output connector 46a. Connector pins P3 and P4 are connected to the relay switching circuit 45 respectively via connector pins P3′ and P4′ of the output connector 46a.

Moreover, connector pins P5 and P6, which are connected to an activation switch 20 provided in the handpiece 2, are connected to connector pins P5′ and P6′ on the side of the output connector 46b, respectively.

In the example of FIG. 4, the connector pin P6′ is grounded, and the connector P5′ is connected to the CPU 42. In this case, the connector P5′ is pulled up to H level, for example, by a resistor R1. Then, when the activation switch 20 is turned on, the level of the connector P5′ is changed from H level to L level, and the CPU 42 senses that an activation switch 20 is turned on. The CPU 42 transmits the signal that is turned on to the CPU 28 of the high-frequency output apparatus 3 via the communication cable 5 so that a high-frequency signal is outputted, and outputs an ultrasound driving signal.

Specifically, the ultrasound cable 9 is connected to the relay switching circuit 45 shown in FIG. 3B via the output connector 46a. In contrast to this, the high-frequency cable 10 is electrically connected to the relay switching circuit 25 inside the high-frequency output apparatus 3 via the output connector 46a and via the docking connector as a connection section between the ultrasound output apparatus 4 and the high-frequency output apparatus 3.

Thus, turning on the activation switch 20 that performs instruction operation of simultaneous output of high-frequency wave and ultrasound will result in that ON information of the activation switch 20 is transferred to the CPU 28 of the high-frequency output apparatus 3 from the CPU 42 of the ultrasound output apparatus 4 via the communication cable 5, and a high-frequency signal and an ultrasound driving signal are simultaneously outputted to the handpiece 2a.

Connector pins P7 and P8 are pins for connection sensing, and are respectively connected to connector pins P7′ and P8′ of the side of the output connector 46a. The connector pins P7′ and P8′ are connected to the HP connector connection-sense circuit 51. Moreover, a resistor R2 is provided between the connector pins P7 and P8.

Then, the HP connector connection-sense circuit 51 performs connection sensing of whether or not the HP connector 15a is connected to the output connector 46a by detecting the resistance value of the resistor R2, and senses what type of handpiece 2a is connected based on the resistance value of the resistor R2.

Similarly, connector pins P9 and P10, which are pins for connection sensing of the HP connector 15b, are connected to connector pins P9′ and P10′ on the side of the output connector 46b. Moreover, a resistor R3 is provided between the connector pins P9 and P10. Note that for the HP connector 15b, connector pins other than the connector pins P9 and P10 which are pins for connection sensing are omitted from illustration.

The connector connection-sense circuit 51 performs connection sensing of whether or not the HP connector 15b is connected to the output connector 46b by detecting the resistance value of the resistor R3, and senses what type of handpiece 2b is connected based on the resistance value of the resistor R3.

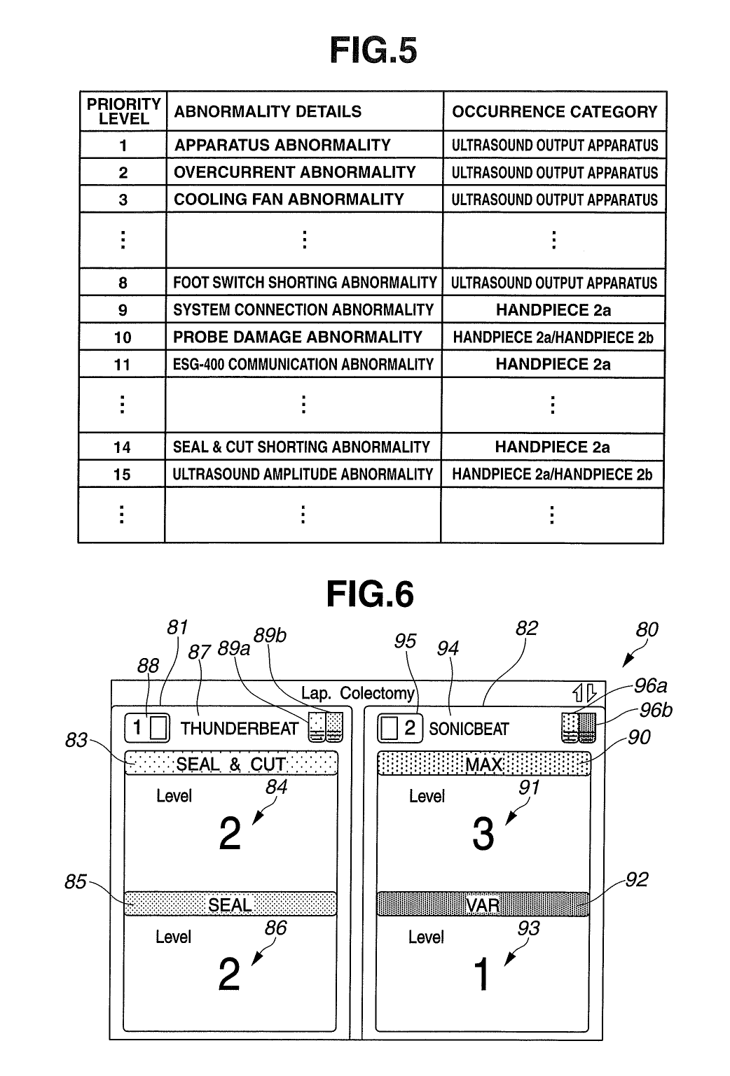

FIG. 5 is a diagram to illustrate the relationships among priority levels, abnormality details, and occurrence categories.

As shown in FIG. 5, an apparatus abnormality, whose priority level is 1, is an abnormality that occurs in the main body of the ultrasound output apparatus 4. Moreover, a probe damage abnormality, whose priority level is 10, is an abnormality that occurs in each of the handpieces 2a and 2b. Further, an ESG-400 communication abnormality, whose priority level is 11, is an abnormality that occurs only in the handpiece 2a.

Here, a display screen which is displayed on the display section 60 when the handpieces 2a and 2b are connected to the ultrasound output apparatus 4 is described.

FIG. 6 is a diagram to illustrate an example of the display screen when the handpieces 2a and 2b are connected to the ultrasound output apparatus 4.

The display screen shown in FIG. 6 is an all-instruments display screen 80 which is automatically displayed on the display section when the handpieces 2a and 2b are connected to the ultrasound output apparatus 4.

The all-instruments display screen 80 includes a first display screen 81 that displays the setting information of the handpiece 2a, and a second display screen 82 that displays the setting information of the handpiece 2b.

The first display screen 81 includes a mode information display section 83, an output level display section 84, a mode information display section 85, an output level display section 86, a model-name display section 87, a symbol icon 88, and FSW connection-sense icons 89a and 89b.

Moreover, the second display screen 82 includes a mode information display section 90, an output level display section 91, a mode information display section 92, an output level display section 93, a model-name display section 94, a symbol icon 95, and FSW connection-sense icons 96a and 96b.

A display of “SEAL & CUT” is made on the mode information display section 83 corresponding to the SEAL & CUT mode. The present output level is displayed on the output level display section 84. Moreover, a display of “SEAL” is made on the mode information display section 85 corresponding to the SEAL mode. The output level that is currently set is displayed on the output level display section 86.

The model name of the handpiece 2a connected to the ultrasound output apparatus 4 is displayed on the model-name display section 87. In FIG. 6, for example, THUNDERBEAT (registered trade mark) is displayed as a model name corresponding to the handpiece 2a on the model-name display section 90.

A symbol similar to the above described symbol 65a is displayed as an icon on the symbol icon 88. Moreover, this symbol is also provided on the grasping portion 6 of the handpiece 2a as described above. Thereby, a user can recognize that the setting information displayed on the first display screen 81 is the setting information of the handpiece 2a by confirming the symbol icon 88.

FSW connection-sense icons 89a and 89b are icons that are displayed when the FSW connector connection-sense section 53 senses that a foot switch not shown is connected to each of the FSW connectors 52a and 52b. Moreover, an FSW connection-sense icon 92a and the mode information display section 83 are displayed in the same color, and an FSW connection-sense icon 92b and the mode information display section 87 are displayed in the same color.

Thus, a user can perform the changing of output level or the like by confirming the FSW connection-sense icons 92a and 92b.

Note that since the configuration of the second display screen 82 is similar to that of the first display screen 81, description thereof will be omitted.

As so far described, it is configured that when the handpieces 2a and 2b are connected to the ultrasound output apparatus 4, the setting information of the handpiece 2a is displayed on the first display screen 81 and the setting information of the handpiece 2b is displayed on the second display screen 82.

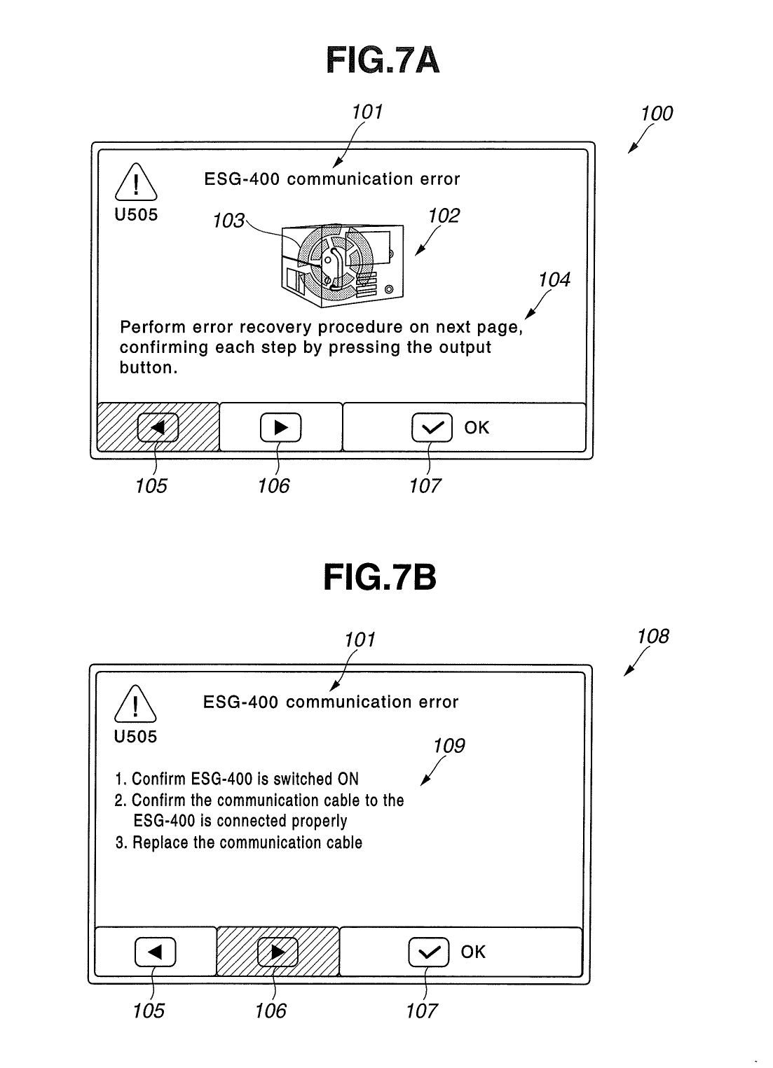

FIG. 7A is a diagram to illustrate an example of the error display screen which is displayed on the display section 60 when an ESG-400 communication abnormality occurs in the handpiece 2a; and FIG. 7B is a diagram to illustrate an example of the countermeasure information display screen which is displayed on the display section 60.

The display screen shown in FIG. 7A shows an error display screen 100 which is displayed on the display section 60 when an ESG-400 communication abnormality occurs in the handpiece 2a. The ESG-400 communication abnormality is an abnormality that occurs only in the handpiece 2a, and the priority level thereof is 11.

The error display screen 100 includes an abnormality details display section 101, an abnormal apparatus display section 102, an abnormal spot display marker 103, a message display section 104, a backward button 105, a forward button 106, and an OK button 107.

The details of an abnormality that has occurred is displayed on the abnormality details display section 101, and an apparatus in which an abnormality has occurred is displayed on the abnormal apparatus display section 102.

Moreover, the abnormal spot display marker 103 is a marker for displaying where in the abnormal apparatus, in which an abnormality has occurred, the abnormality is located. The abnormal spot display marker 103, which is given a predetermined color, lights up or blinks to notify the abnormal spot to the user.

A message for the abnormality that has occurred is displayed on the message display section 104.

The backward button 105 is a button for moving backward to the previous display screen, and the forward button 106 is a button for moving forward to the next display screen.

Further, the OK button 107 is a button for closing the error display screen 100 and displaying the all-instruments display screen 80 shown in FIG. 6.

A user can move to a countermeasure information display screen 108 shown in FIG. 7B by pressing the forward button 106. The countermeasure information display screen 108 includes a countermeasure information display section 109. Information for coping with the abnormality that has occurred is displayed on the countermeasure information display section 109. A user can move backward to the error display screen 100 shown in FIG. 7A by pressing the backward button 105.

In this way, when an abnormality occurs in the handpiece 2a, a user may continue treatment by using the handpiece 2b after moving backward to the all-instruments display screen 80 by pressing the OK button 107.

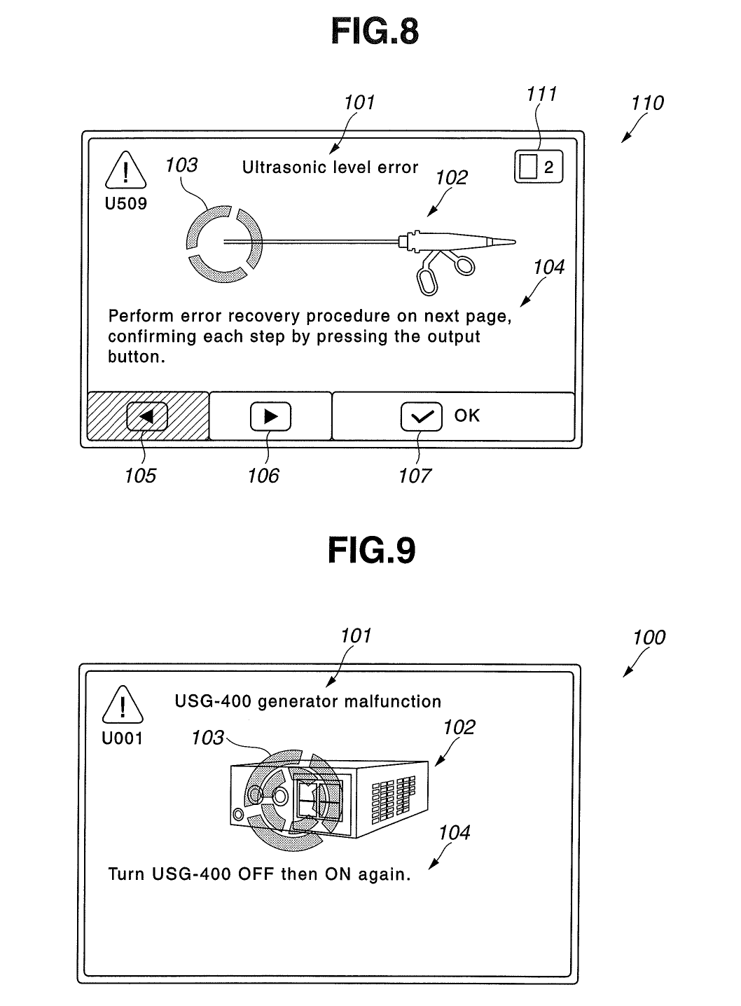

Then, when a new ultrasound amplitude abnormality occurs while the user is giving treatment by using the handpiece 2b, the error display screen shown in FIG. 8 is displayed on the display section 60.

FIG. 8 is a diagram to illustrate an example of the error display screen which is displayed on a display section 60 when an ultrasound amplitude abnormality occurs in the handpiece 2b.

The display screen shown in FIG. 8 shows an error display screen 110 which is displayed on the display section 60 when an ultrasound amplitude abnormality occurs in the handpiece 2b. As shown in FIG. 5, the ultrasound amplitude abnormality is a type of abnormality that may occur in both the handpieces 2a and 2b, and the priority level thereof is 15.

The priority level of the ultrasound amplitude abnormality is 15, which is lower than the priority level of the ESG-400 communication abnormality described above.

Conventionally, there is a risk of overlooking an abnormality that has occurred in the handpiece 2b being used, because it is arranged that the abnormality information of the abnormality of higher priority level is always displayed on the display section so that the information of the abnormality that has occurred in the handpiece 2b is not displayed.

In contrast, the present embodiment is configured such that regarding abnormalities that have simultaneously occurred in different medical devices, that is, the handpieces 2a and 2b, the abnormality information of the abnormality that occurs later is displayed on the display section 60 regardless of the priority level. Thereby, a user is prevented from overlooking an abnormality that occurs in the handpiece 2b in use.

Moreover, a symbol icon 111 is displayed in the upper right facing FIG. 8 in the error display screen 110. As described so far, a symbol similar to the symbol icon 111 is provided in the grasping portion 6 of the handpiece 2b.

A plurality of (two, in this case) handpieces 2a and 2b are connected to the ultrasound output apparatus 4. Moreover, the ultrasound amplitude abnormality is an abnormality which can occur in both the handpieces 2a and 2b. Therefore, when an ultrasound amplitude abnormality occurs, it becomes difficult for a user to recognize in which of the handpieces 2a and 2b an abnormality has occurred.

Nevertheless, displaying the symbol icon 111 in the error display screen 110 as shown in FIG. 8 allows the user to recognize in which of the handpieces an abnormality has occurred.

FIG. 9 is a diagram to illustrate an example of the error display screen which is displayed on the display section 60 when an apparatus abnormality occurs in the ultrasound output apparatus 4.

The display screen shown in FIG. 9 shows an error display screen 120 which is displayed on the display section 60 when an apparatus abnormality occurs in the ultrasound output apparatus 4. The apparatus abnormality is an abnormality that occurs in the ultrasound output apparatus 4 as shown in FIG. 5 and the priority level thereof is 1.

This error display screen 120 is configured not to include the backward button 105, the forward button 106, and the OK button 107 in FIG. 7A. When an abnormality occurs in the ultrasound output apparatus 4, the handpieces 2a and 2b become unusable. For that reason, it is configured not to include the OK button 107 such that the error display screen 120 cannot be closed.

Moreover, an abnormality that occurs in the ultrasound output apparatus 4 is a type of abnormality whose priority level is high and which makes the handpieces 2a and 2b unusable. For that reason, the present embodiment is configured such that when an abnormality occurs in the ultrasound output apparatus 4, the error display screen for the abnormality that has occurred in the ultrasound output apparatus 4 is preferentially displayed even in the case that, for example, the error display screen of an abnormality that has occurred in the handpieces 2a or 2b is being displayed on the display section 60.

Moreover, in the case of a foot switch shorting abnormality whose priority level is 8 as well, the OK button will not be displayed on the error display screen when an abnormality occurs, because it is a type of abnormality that occurs in the ultrasound output apparatus 4. However, this foot switch shorting abnormality is an abnormality liable to be caused by an operation mistake by a user. For that reason, it may be configured such that the OK button is displayed on the error display screen when an error state is resolved so that the error display screen can be closed.

Further, the present embodiment is configured such that when a plurality of abnormalities occur simultaneously in the same occurrence category, the error display screen of an abnormality of higher priority level is displayed on the display section 60.

For example, when an apparatus abnormality whose priority level is 1 and a cooling fan abnormality whose priority level is 3 occur simultaneously in the ultrasound output apparatus 4, the error display screen of the apparatus abnormality whose priority level is higher is displayed on the display section 60. Similarly, when a probe damage abnormality whose priority level is 10 and an ultrasound amplitude abnormality whose priority level is 15 occur simultaneously in the handpiece 2a, the error display screen of the probe damage abnormality whose priority level is higher is displayed on the display section 60. The same applies with the handpiece 2b.

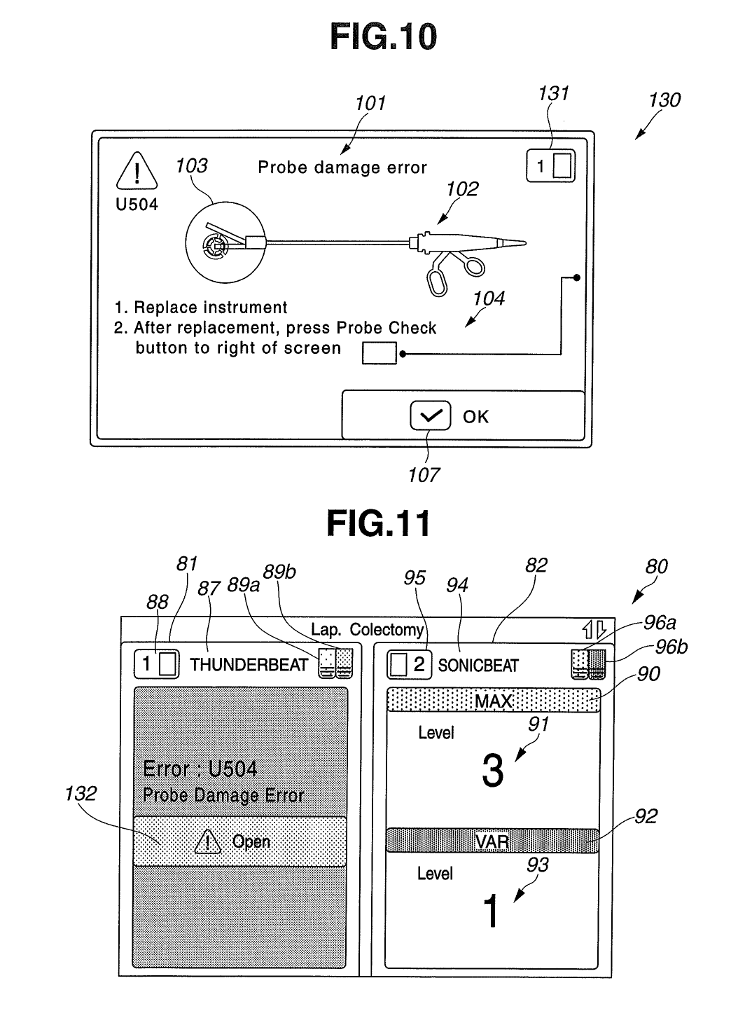

FIG. 10 is a diagram to illustrate an example of the error display screen which is displayed on the display section 60 when a probe damage abnormality occurs in the handpiece 2a.

The display screen shown in FIG. 10 shows an error display screen to be displayed on the display section 60 when a probe damage abnormality occurs in the handpiece 2a. Here, since the abnormality has occurred in the handpiece 2a, a symbol similar to the symbol 65 is displayed as a symbol icon 131 on the error display screen 130.

Further, although it is possible to move backward to the all-instruments display screen 80 of FIG. 6 by pressing the OK button 107, when a probe damage abnormality occurs, the corresponding display screen is blacked out.

This is because continuing the ultrasound output while a probe damage abnormality has occurred may lead to falling off of the probe. Therefore, the present embodiment is configured such that when a probe damage abnormality occurs, the use of the handpiece in which the abnormality has occurred is inhibited, and the setting information of the handpiece in which the abnormality has occurred cannot be confirmed or changed.

FIG. 11 is a diagram to illustrate an example of an all-instruments display screen 80 in which setting information is blacked out.

As shown in FIG. 11, when a probe damage abnormality occurs in the handpiece 2a, the setting information of the first display screen corresponding to the handpiece 2a is blacked out, and the first display screen 81 turns into a display state indicating that the output is disabled. Moreover, an open button 132 is provided in the first display screen 81, and a user can open the error display screen 130 shown in FIG. 10 by pressing the open button 132 to confirm the details of error.

Next, such processing to cause an error display screen to be displayed on the display section 60 will be described.

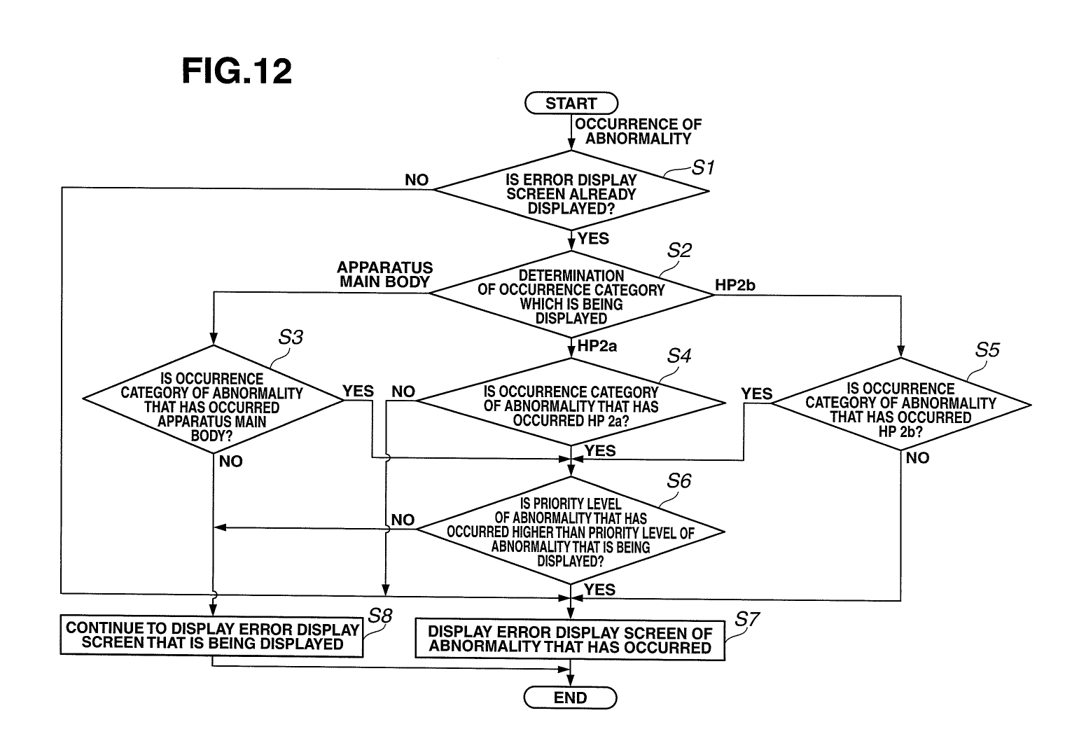

FIG. 12 is a flowchart to illustrate an example of the flow of the processing to cause an error display screen to be displayed on the display section 60.

First, upon occurrence of an abnormality, it is determined whether or not an error display screen is already displayed on the display section 60 (step S1). When the error display screen is not being displayed, the determination result is NO and the process proceeds to step S7. In the meantime, when the error display screen is already displayed, the determination result is YES and the determination of the occurrence category which is being displayed is performed (step S2). When the occurrence category is the apparatus main body, that is, the ultrasound output apparatus 4, the process proceeds to step S3; when the occurrence category is the handpiece 2a, the process proceeds to step S4, and when the occurrence category is the handpiece 2b, the process proceeds to step S5.

Then, it is determined whether or not the occurrence category of the abnormality that has occurred is the apparatus main body (step S3). When the occurrence category of the abnormality that has occurred is the apparatus main body, the determination result is YES, and the process proceeds to step S6. In the meantime, when the occurrence category of the abnormality that has occurred is not the apparatus main body, the determination result is NO and the process proceeds to step S8. Further, it is determined whether or not the occurrence category of the abnormality that has occurred is the handpiece 2a (step S4). When the occurrence category of the abnormality that has occurred is the handpiece 2a, the determination result is YES, and the process proceeds to step S6. In the meantime, when the occurrence category of the abnormality that has occurred is not the handpiece 2a, the process proceeds to step S7. Furthermore, it is determined whether or not the occurrence category of the abnormality that has occurred is the handpiece 2b (step S5). When the occurrence category of the abnormality that has occurred is the handpiece 2b, the determination result is YES, and the process proceeds to step S6. In the meantime, when the occurrence category of the abnormality that has occurred is not the handpiece 2b, the process proceeds to step S7.

When the determination result is YES in steps S3, S4, and S5, that is, abnormalities have occurred in the same occurrence category, it is determined whether or not the priority level of the abnormality that has occurred is higher than the priority level of the abnormality that is being displayed (step S6). When the priority level of the abnormality that has occurred is higher than the priority level of the abnormality that is being displayed, the determination result is YES and an error display screen of the abnormality that has occurred is displayed on the display section 60 (step S7), thereby ending the process. In the meantime, when the priority level of the abnormality that has occurred is lower than the priority level of the abnormality that is being displayed, the determination result is NO and the error display screen that is being displayed continues to be displayed (step S8), thereby ending the process.

Moreover, when it is determined that an error display screen is not being displayed in step S1, an error display screen of the abnormality that has occurred is displayed on the display section 60 in step S7.

Further, when it is determined that the occurrence category of the abnormality that has occurred is not the apparatus main body in step S3, the error display screen which is being displayed continues to be displayed in step S8.

Furthermore, when it is determined that the occurrence category of the abnormality that has occurred is not the handpiece 2a in step S4, and it is determined that the occurrence category of the abnormality that has occurred is not the handpiece 2b in step S5, an error display screen of the abnormality that has occurred later is displayed on the display section 60 regardless of the priority level in step S7.

As so far described, the GUI control section 57 of the ultrasound output apparatus 4 is configured such that when abnormalities occur simultaneously in different medical devices, that is, the handpieces 2a and 2b, an error display screen of the abnormality that occurs later is displayed on the display section 60 regardless of the priority level. As a result of this, a user can easily recognize abnormalities that occur in different medical devices.

Note that it is configured, as described above, such that turning on the power supply button 67 while simultaneously pressing the probe check button 63 and the menu button 64 of the sheet button 61 allows a direct transition to the screen of various log data. The various log data is saved as backup data in the memory 56.

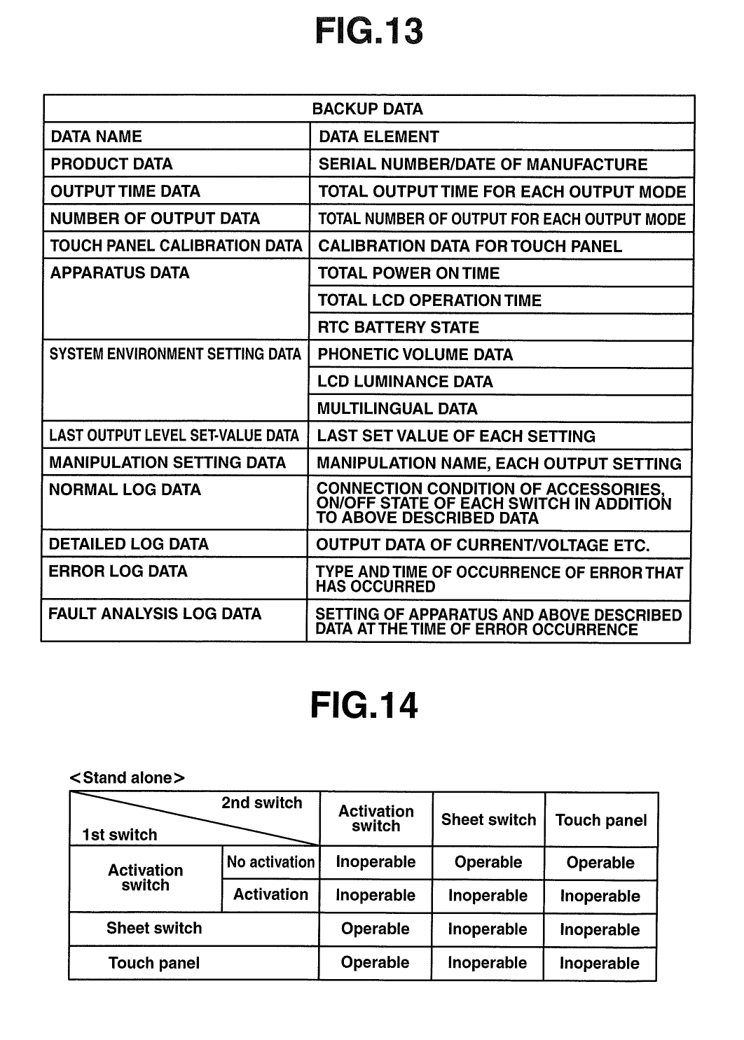

FIG. 13 is a diagram to illustrate an example of the backup data.

As shown in FIG. 13, the backup data includes, for example, detailed log data and error log data as the above described various log data. Moreover, the backup data includes product data and output time data, etc. besides the various log data. The product data includes data such as a serial number and the date of manufacture, which are saved in the memory 56 as data elements. Moreover, the output time data includes data such as a total output time for each output mode, which is saved in the memory 56. In this way, the display section 60 can display the information of those backup data.

Moreover, the ultrasound output apparatus 4 and the high-frequency output apparatus 3 of the present embodiment have a switching rule as described below.

FIG. 14 is a diagram to illustrate an example of the switching rule to perform output operation of the ultrasound output apparatus or the high-frequency output apparatus in the case of standalone operation in the present embodiment.

As shown in FIG. 14, in the case of standalone as an environment in which the apparatus operates independent of other instruments, as shown in FIG. 14, there are three types of switches including an activation switch, a sheet switch, and a touch panel. A first-press priority rule is applied to each switch.

Even when the sheet switch or the touch panel is being pressed, the activation switch is operable. Moreover, when the activation switch is pressed, and an ultrasound or high-frequency wave is being outputted, the sheet switch and the touch panel will be inoperable.

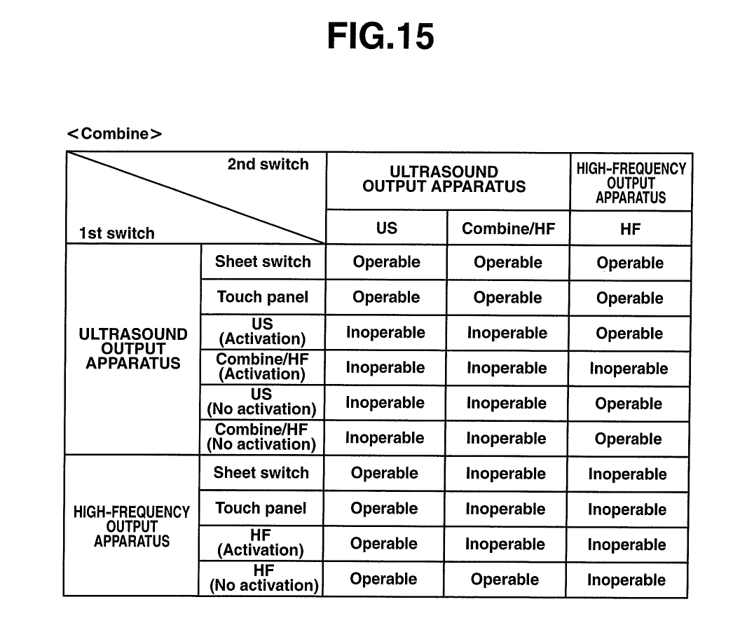

FIG. 15 is a diagram to illustrate an example of the switching rule relating to the output operation in the case of combined operation of the ultrasound output apparatus and the high-frequency output apparatus in the present embodiment. Note that “Combine/HF” refers to an HF switch for operating in combination with the ultrasound output apparatus.

As shown in FIG. 15, the activation switch (US and Combine/HF) is operable even when the sheet switch or the touch panel is being pressed. Moreover, the activation switch (HF) is inoperable when the sheet switch or the touch panel is being pressed.

Moreover, exclusive control according to the presence/absence of output is performed between system instruments. Further, exclusive control based on the activation switch will not be performed between system instruments.

As so far described, in the case of combined operation, communication is performed between system instruments; output control is performed as shown in FIG. 15; and judgment on whether or not output can be performed from the states of peripheral instruments.

Note that in the execution of each step in the flowchart in the present description, the execution order may be changed, or a plurality of the steps may be executed simultaneously, or the execution order may be changed for each execution, as long as execution does not contradict the nature of each step.

The present invention will not be limited to the embodiment described above, and may be subjected to various changes and modifications within a scope not changing the spirit of the present invention.

What is claimed is:

1. A medical apparatus connectable with a plurality of medical devices, the medical apparatus comprising:
a priority level judgment section for judging a priority level for preferentially notifying a user of a plurality of abnormalities that occur in the plurality of medical devices;
a display control section for causing abnormality information whose priority level judged by the priority level judgment section is high to be displayed on a display section; and
a priority level changing section, which upon detecting an occurrence of an abnormality from a medical device other than the medical device in which the abnormality information being displayed on the display section is originated, causes abnormality information from the medical device to be displayed on the display section in place of the abnormality information being displayed on the display section regardless of the priority level of the abnormality.

2. The medical apparatus according to claim 1, wherein
the display section can display abnormality information that occurs in the medical apparatus, and
when an abnormality occurs in the medical apparatus while an abnormality is occurring in any of the plurality of medical devices, the priority level changing section causes the abnormality information that occurs in the medical apparatus to be displayed on the display section.

3. The medical apparatus according to claim 1, wherein
when a medical device that is different from the medical device in which abnormality information being displayed on the display section is originated is operated, the priority level changing section causes the abnormality information being displayed on the display section not to be displayed on the display section.

4. The medical apparatus according to claim 1, wherein
the medical apparatus is an ultrasound output apparatus that outputs an ultrasound driving signal to the plurality of medical devices.

5. The medical apparatus according to claim 1, wherein
the plurality of medical devices are medical handpieces, respectively.

6. The medical apparatus according to claim 5, wherein
when an abnormality that occurs in any of the plurality of medical handpieces is an ultrasound probe abnormality, the priority level changing section causes the display screen on which setting information of the medical handpiece in which the abnormality occurs is displayed to turn into a display state indicating that output is disabled.
